(19) United States
(12) Patent Application Publication
(10) Pub. No.: US 2026/0123968 A1
(43) Pub. Date: May 7, 2026

(54) DEVICES AND METHODS FOR BONE DISTRACTION, COMPRESSION, & ROTATION

(71) Applicant: Acumed LLC, Hillsboro, OR (US)

(72) Inventors: Brayden Reiland, Portland, OR (US); Brian Conley, Portland, OR (US); Caleb Martin, Beaverton, OR (US)

(21) Appl. No.: 19/381,514

(22) Filed: Nov. 6, 2025

Related U.S. Application Data
(60) Provisional application No. 63/717,608, filed on Nov. 7, 2024.

(51) Int. Cl. A61B 17/80; A61B 17/00; A61B 17/68; A61B 90/00

(52) U.S. Cl. CPC A61B 17/8019; A61B 90/08; A61B 2017/00407; A61B 2017/681; A61B 2090/0807

(57) ABSTRACT

Provided herein are methods and systems for the compression and/or distraction of a bone fracture. The system includes a rod configured to be at least partially inserted into the bone and a body defining a bore configured to circumscribe a portion of the rod. The system further includes a device with a first member and a second member joined at a fulcrum. The first member includes a channel for receiving a portion of the rod. The bone fracture compression or distraction includes inserting the rod into the bone, placing the first member of the device over the rod, positioning the second member of the device into a feature of a bone plate, and moving the first member and the second member toward each other.

### INCORPORATION BY REFERENCE TO ANY PRIORITY APPLICATIONS

This application claims benefit of U.S. Provisional Ser. No. 63/717,608, filed Nov. 7, 2024 and entitled “DEVICE AND METHOD FOR BONE DISTRACTION AND COMPRESSION,” the disclosure of which is hereby incorporated by reference and made part of this specification as if set forth fully herein in its entirety. Any and all applications for which a foreign or domestic priority claim is identified in the Application Data Sheet as filed with the present application are hereby incorporated by reference under 37 CFR 1.57.

### BACKGROUND

### Field

The present disclosure provides systems and methods for aligning a fracture that prevents drawbacks associated with current methods.

### Description of the Related Art

After a bone fracture, bone fragments may require realignment to ensure the correct positioning for healing. Typically, for proper alignment, a bone plate is secured on the bone or bones to hold the fracture in place while healing. The plate may extend over multiple bone fragment of the same bone or multiple bones around a joint or joints (e.g., a wrist spanning plate to address distal radius fractures).

Alignment of the bone fracture may include compression, distraction, or rotation of the bone fracture. Compression includes moving the bone fragments toward each other while distraction includes moving the bone fragments away from each other, such as to restore the length of the bone.

### SUMMARY

The present disclosure provides new and innovative devices, systems, and methods for aligning a bone fracture that avoids problems associated with the current solutions, such as the screw for distraction/compression being too long initially and needing to be shortened, possibility of damaging and stripping of a screw inserted into the bone plate or damage to the bone around the screw, and/or tendency for the bone distractor device to disengage from the screw during use. The devices, systems, and methods disclosed herein can additionally be used for more diverse types of fracture fixation, such as wrist spanning, etc. Alignment of the bone fracture as disclosed herein may include compression, distraction, or rotation of the bone fracture. Compression includes moving the bone fragments toward each other while distraction includes moving the bone fragments away from each other, such as to restore the length of the bone. Alignment of bone fracture as disclosed herein may alternatively or additionally include stretching of soft tissue such as but not limited to ligaments (e.g., between multiple bones), which may or may not involve moving of the bone fragments relative to each other.

Current compression and distraction tools may have a plier design including a first handle and a second handle connected at a fulcrum. The first handle may include a channel for a driver to extend therethrough. The driver can engage a head of a screw inserted into the bone via an aperture of a bone plate. The second handle may extend to a tip that can engage another aperture of the bone plate. Pivotal movements of the two handles at the fulcrum can result in compression or distraction of the bone fragments over which the plate extends. The screw that is used for compression/distraction needs to be of sufficient length, e.g., a screw that can achieve bicortical purchase when unlocked to the plate, to have a secure grip or hold in the bone to allow the force from the tool to be transmitted to the bone fragment or bone to result in compression, distraction, and/or rotation. However, to achieve bicortical purchase when the screw is unlocked, a user may tend to over-size the screw (e.g., choosing a longer screw) used for distraction/compression. The screw may be too long when locked into the plate and may then need to be shortened (e.g., by being replaced with a shorter screw). As a result, in some implementations, a user of the current compression and distraction tool may need to first insert a temporary screw (e.g., a longer screw) to effect the alignment of the fracture, remove the temporary screw while maintaining the alignment (which can be difficult), and insert a permanent screw (e.g., a shorter screw).

A challenge of the current compression and distraction tools engaging the temporary screw is possible stripping and damaging of the screw. In some implementations, it can be difficult to remove the temporary screw if the screw was stripped or damaged. Bone and other tissue around the bone may also be at risk of damage during engagement between the tool and the temporary screw and/or during removal of the temporary screw. Another challenge is the tendency for the driver of the current compression and distraction tool to disengage from the screw head, such as when a non-axial or side load is applied. It can be inconvenient and time-consuming if the user needs to re-engage the screw during use. The disengagement of the screw from the driver can also make it more prone to damage or strip the screw. Yet another challenge is to maintain the alignment when the temporary screw is removed from the bone and the plate.

The devices, systems, and methods provided herein use a rod that is configured to be temporarily inserted into the bone (e.g., instead of separately inserting a temporary screw) and that extends above a surface of the bone where the bone plate is positioned or be partially inserted into another bone separate from the bone covered by the plate. The temporary rod provides one point of fixation for alignment of the bone fracture, while a plier like device provides a second point of fixation. The rod directly inserted into the bone can avoid the problem of damaging or stripping the temporary screw and disengagement of the driver from the screw. The device can further include a mechanism for compressing the bone plate against the bone, facilitating maintenance of alignment of the bone fragments or fracture.

One aspect of the present disclosure provides a system for realignment of a bone fracture, such as distraction, compression, and/or rotation of bone or bone fragments, and/or stretching of soft tissue. In some embodiments, the system includes a rod configured to be inserted into the bone, a body defining a bore configured to circumscribe a portion of the rod, and a plier like device. The plier like device includes a first member and a second member joined at a fulcrum. One of the first member or the second member defines a channel for receiving at least a portion of the rod.

Another aspect of the present disclosure provides a method for alignment of a bone fracture, such as compression, distraction, or rotation of bone or bone fragment, and/or stretching of soft tissue using the system disclosed herein. In some embodiments, the method includes inserting the rod into the bone, placing the first member over the rod, and positioning the second member into a feature of the bone plate. The rod may be inserted in the plate or outside the plate on the same bone where the plate is extended over in any suitable location (e.g., proximal to the plate, distal to the plate, lateral to the plate, medial to the plate, etc.), or inserted in a different bone in any suitable location. A fastener may be used to couple the plate to the bone prior to distracting, compressing, or rotating the bone or bone fragment. Movement of the first member and the second member towards (or away from) each other provides distraction or compression. In order to secure the bone plate to the bone and keep the alignment of the fracture, in some embodiments, the method includes translating the body along the rod to compress the bone plate against the bone. Alternatively, the alignment can be kept during removal of the rod by a k-wire, a clamp, a screw, etc. The method may include fixing the bone plate to the bone using one or more fasteners for healing of the fracture.

Additional features and advantages of the disclosed methods are described in, and will be apparent from, the following Detailed Description and the Figures.

### BRIEF DESCRIPTION OF THE DRAWINGS

FIG. 1 illustrates a system for alignment of a bone fracture according to an example of the present disclosure.

FIG. 2 illustrates the plier like device of the system of FIG. 1 according to an example of the present disclosure.

FIG. 3 illustrates a top view of a ratcheting mechanism of the plier like device of FIG. 2 according to an example of the present disclosure.

FIG. 4 illustrates a plier like device according to another example of the present disclosure.

FIG. 5 illustrates a rod of the system of FIG. 1 according to an example of the present disclosure.

FIGS. 6 and 7 illustrate a body of the system of FIG. 1 according to an example of the present disclosure.

FIGS. 8, 9A, and 9B illustrate bodies of the system of FIG. 1 according to alternative examples of the present disclosure.

FIGS. 10 and 11 illustrate the rod and the body of the system of FIG. 1 according to an example of the present disclosure.

FIGS. 12, 12A, 13, 13A, 14, 14A, 15, and 15A illustrate a method of aligning a bone fracture using the system illustrated in FIG. 1 according to an example of the present disclosure in which a rod of the system is on the plate.

FIG. 16 illustrate a method of aligning a bone fracture using the system disclosed herein according to an example of the present disclosure in which a rod of the system is on the plate.

FIG. 17 illustrates a method of aligning a bone fracture using the system disclosed herein according to an example of the present disclosure in which a rod of the system is not on the plate and not on the same bone as the plate.

FIG. 18 illustrates a method of aligning a bone fracture using the system disclosed herein according to an example of the present disclosure in which a rod of the system is not on the plate but on the same bone as the plate.

FIG. 19 illustrates a method of aligning a bone fracture using the system disclosed herein according to an example of the present disclosure in which a rod of the system is not on the plate but on the same bone as the plate.

### DETAILED DESCRIPTION

The present disclosure is directed to devices, systems and methods for aligning a bone fracture. In some embodiments, aligning a bone fracture includes one or more of distraction, compression, and/or rotation of bone fragments, bones, and/or soft tissue (e.g., across joint(s)). Throughout the present disclosure, proximal and distal are used in reference to the patient's body or torso. For example, the end of an instrument closer to the patient's body is a proximal end and the opposite end of the instrument, which is further away from the patient's body is the distal end. As another example, an implant that is inserted closer to the patient's torso is more proximal than another implant that is inserted further away from the patient's torso.

### Overview of System

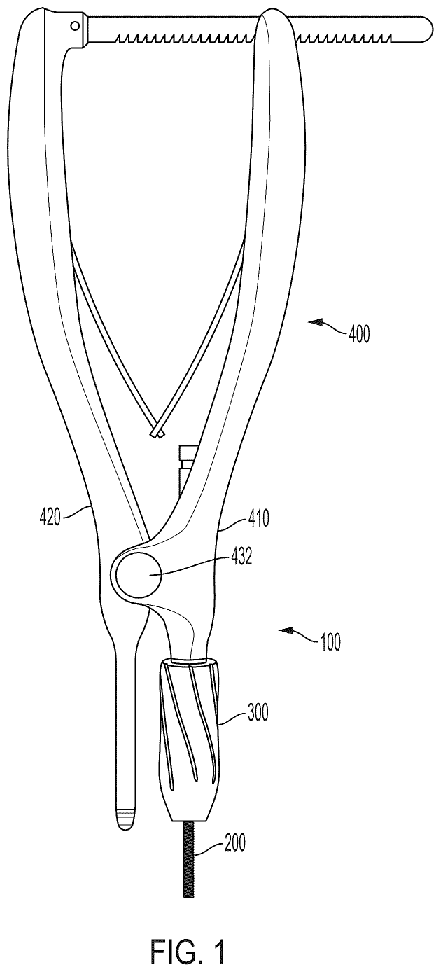

FIG. 1 illustrates a system 100 for aligning a bone fracture according to an example of the present disclosure. In a preferred embodiment, the system 100 includes a rod 200, a body 300 defining a bore configured to receive the rod 200, and a plier like device 400. The plier like device 400 includes a first member 410 and a second member 420 joined together at a fulcrum 432. In some embodiments, one of the first member 410 or the second member 420 includes a channel for receiving a portion of the rod 200.

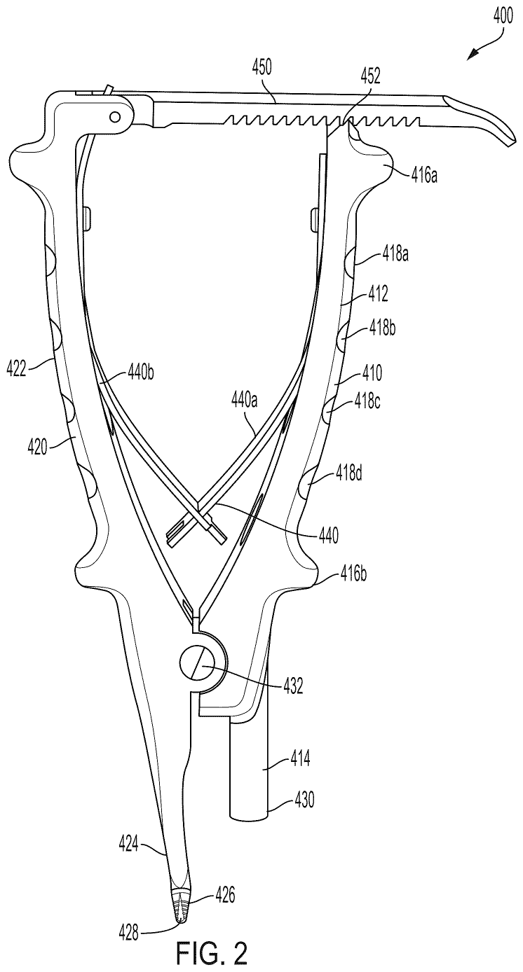

FIG. 2 illustrates an example of the plier like device 400 according to an example of the present disclosure. As described previously, in the depicted embodiment, the plier like device 400 includes a first member 410 and a second member 420 joined together at a fulcrum 432. The first member 410 and the second member 420 may pivot about the fulcrum 432. In some embodiments, the first member 410 may be shorter than the second member 420. However, in some embodiments, the first member 410 may be longer than or the substantially the same length as the second member 420.

In some embodiments, each of the first member 410 and the second member 420 include a first end portion 412, 422 on one side of the fulcrum 432 and a second end portion 414, 424 on the other side of the fulcrum 432. The first end portions 412, 422 may comprise a handle or a grip to be grasped by a user. For example, in some embodiments, the first end portions 412, 422 may include one or more notches 416a, 416b to fit a user's hand while using the device. Notch 416b may prevent a user's hand from slipping while using the device 400. While FIG. 2 shows two notches 416a, 416b, it can be appreciated that one notch or any number of notches may be used. Additionally or alternatively, in some embodiments, the first end portions 412, 422 may include one or more cut outs (i.e. 418a, 418b, 418c, 418d) configured to hold a user's fingers.

The second end portions 414, 424 may be configured to interface, either directly or indirectly, with the bone or a bone plate to provide distraction, compression, or rotation, or any combinations thereof. In some embodiments, movement of the first end portions 412, 422 together causes the second end portions 414, 424 to move apart from each other and vice versa. In other embodiments, movement of the first end portions 412, 422 together causes the second end portions 414, 424 to move closer to each other and vice versa.

In a particularly preferred embodiment, a second end portion 414, 424 of one member 410, 420 of the plier like device 400 includes a channel 430 through the member 410, 420. In some embodiments, the channel 430 may begin at a second end portion 414, 424 of the member 410, 420, such as at an end of the second end portion 414, 424. In some embodiments, the channel may end at a first end portion of the member or the second end portion. The channel 430 may have a diameter greater than or substantially equal to at least a portion of the rod 200 (i.e. a distal portion of the rod 200, which will be disclosed in greater detail elsewhere in the present disclosure) of the system 100 so the rod 200 can pass through the channel 430. The channel 430 may include a smooth interior surface for receiving the rod 200.

In some embodiments, the second end portion 414, 424 of the other member 410, 420 may be configured to interface with a portion of a bone plate or the bone. In some embodiments, the width of the member 410, 420 tapers from one end of the arm towards the other end. Additionally or alternatively, the end of the member 410, 420 may include one or more grooves 426 or one or more cutouts 428 to help provide grip to the bone plate or the bone. The one or more grooves 426 or the one or more cutouts 428 may also help align the member 410, 420 in a feature of the bone plate. In some embodiments, the one or more cutouts 428 are oriented in a first direction and the one or more grooves 426 are oriented in a second direction, preferably substantially perpendicular to the first direction.

As shown in FIG. 2, in several embodiments, the channel 430 is located on the first member 410. As shown in FIG. 2, in several embodiments, the groove(s) 426 and/or cutout(s) 428 are located on the second member 420.

In some embodiments, the plier like device 400 may include a biasing element 440 between the first member 410 and the second member 420. The biasing element 440 may include a spring (i.e. a leaf spring, a conical spring) or some type of resilient member. The biasing element 440 may act to provide resistance when bringing the first and second member 410, 420 together and may bias the plier like device 400 in an open position. This may result in more controlled distraction or compression. As shown in FIG. 2, in a preferred embodiment, the biasing element 440 may include a first biasing element 440a (e.g., a leaf spring, a torsion spring, etc.) coupled to the first member 410 and a second biasing 440b element (e.g., a leaf spring, a torsion spring, etc.) coupled to the second member 420. The first and the second biasing element 440a, 440b may interface between the first member 410 and the second member 420.

In some embodiments, one of the first or the second members 410, 420 may include a ratcheting element 450. In some embodiments, the ratcheting element 450 extends from one of the first or second members 410, 420 (e.g., rotatably coupled to the second member 420 as shown in FIG. 2) to the other of the first or second members 410, 420 (e.g., the first member 410 as shown in FIG. 2). The other of the first member 410 or the second member 420 may include a ridge 452 configured to interface with a plurality of teeth of the ratcheting element 450, such as to translate along a length of the ratcheting element 450. The ratcheting element 450 and ridge 452 may keep the first member 410 and the second member 420 in a fixed position after movement of the members 410, 420, which may aid in holding the distraction or compression. The configuration may be reversed with the ridge on the second member and the ratcheting element rotatably coupled to the first member. In some embodiments, the reversed configuration may be advantageous when used on a contralateral side of the patient's body, by a left-handed user, etc.

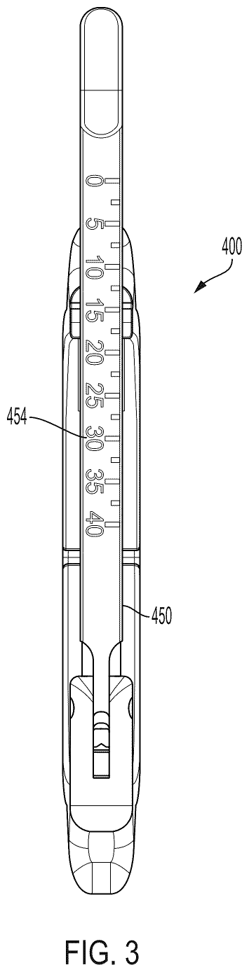

As illustrated in FIG. 3, in some embodiments, the ratcheting element 450 includes one or more markings 454 indicative of the amount of distraction or compression applied to the bone fragments by the system 100. The markings 454 may help a user determine the amount of compression or distraction or if a desired amount of compression or distraction has been reached. The amount of distraction or compression may be determined by looking at the marking aligned when the ridge 452 is in one of the plurality of teeth of the ratcheting element 452. In some embodiments, the markings 454 indicate every 10 mm, every 5 mm, every 2.5 mm, every 1 mm, any of the increments within ranges defined by those values, or any suitable increments of distraction or compression.

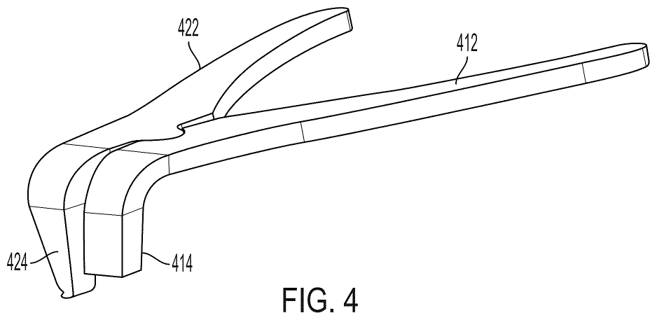

Referring back to FIG. 2, the first end portions 412, 422 define a plane and the second end portions 414, 424 define a plane. In some embodiments, the first end portions 412, 422 and the second end portions 414, 424 are in the same plane as shown in FIG. 2. However, as depicted in FIG. 4, in some embodiments, the plane defined by the first end portions 412, 424 may be at an angle to the plane defined by the second end portions 414, 424. In some embodiments, the plane defined by the first end portions 412, 422 is substantially perpendicular to the plane defined by the second end portions 414, 424. In other embodiments, the plane defined by the first end portions 412, 422 is between about 45 degrees and 90 degrees, between about 90 degrees and 135 degrees, or substantially parallel to the plane defined by the second end portions 424, 414. Positioning the second end portions 414, 424 at an angle relative to the first end portions 412, 422 may allow better visibility of the bone plate during the procedure and may require a smaller incision.

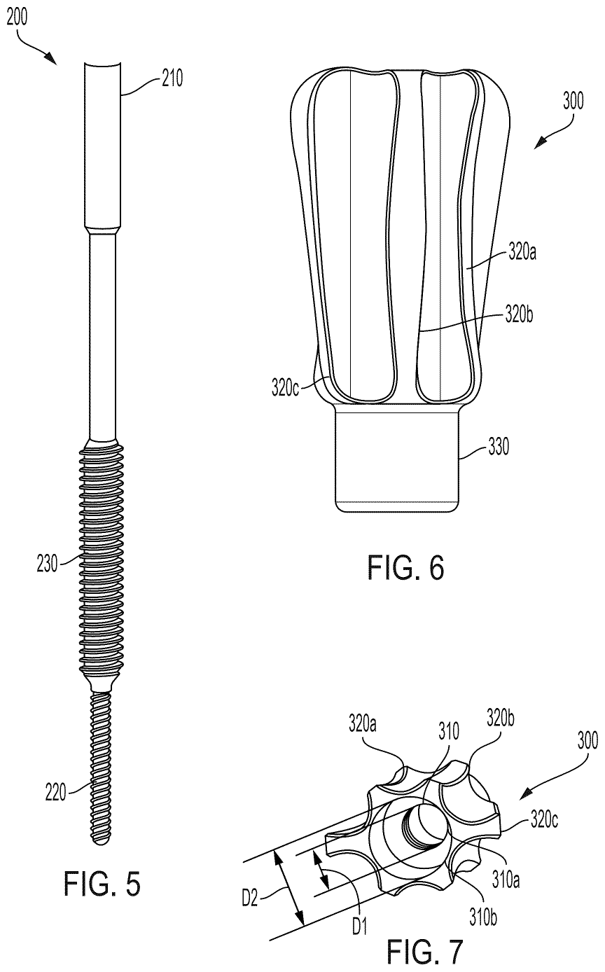

FIG. 5 illustrates a rod 200 according to an example of the present disclosure. The rod 200 is configured to be (at least partially) inserted into a bone to provide an anchor point for distraction and compression. In some embodiments, the rod 200 may be temporarily inserted into the bone and removed after fixation of the bone plate. In some embodiments, the rod 200 is an elongate member with a length greater than a width.

In a particularly preferred embodiment, the rod 200 includes at least two portions. The first portion 220 may extend from the distal end of the rod 200 and the second portion 230 may be more proximal than the first portion 220. As disclosed herein elsewhere, the first portion 220 of the rod 200 may be long enough to provide a stronger anchor point in the bone, e.g., by providing bicortical purchase of the bone. The first portion 220 may be configured to be inserted into the bone while the second portion 230 may be configured to interface with the body 300 and/or the plier like device 400. In some embodiments, the first portion 220 may have a diameter that is smaller than a diameter of a second portion 230 or any other portion of the rod 200. In some embodiments, the second portion 230 may have a diameter that is larger than a diameter of the first portion 220 or any other portion of the rod 200. In an alternative embodiment, the second portion 230 may have a diameter that is substantially the same as a diameter of the first portion 220 or any other portion of the rod 200.

In some embodiments, the first portion 220 of the rod 200 extends along about half, about a third, or about a fourth of the length of the rod 200. In some embodiments, at least a portion of the first portion 220 and/or the second portion 230 may include a thread. In some embodiments, the thread may extend along an entire length of the first portion 220 and/or the second portion 230. In some embodiments, the thread may extend along a portion of the length of the first portion 220 and/or the second portion 230. In some embodiments, the rod 200 may include a thread extending along whole length of the rod. In some embodiments, the thread of the rod 200 comprises a bone thread that supports engagement of the rod in the bone.

In some embodiments, one end (e.g., distal end 210) of the rod 200 may include an interface for engaging a driver, such as a manual driver or a powered driver. The driver interface 210 may include a hexalobe or any other suitable driver interface. In some embodiments, the rod 200 may be sell-drilling, meaning a pilot hole is not required before drilling the rod 200 into the bone. For example, an end (e.g., proximal end) of the rod 200 may include one or more cutting flutes. In some embodiments, the rod 200 may require a pilot hole before drilling the rod 200 into the bone.

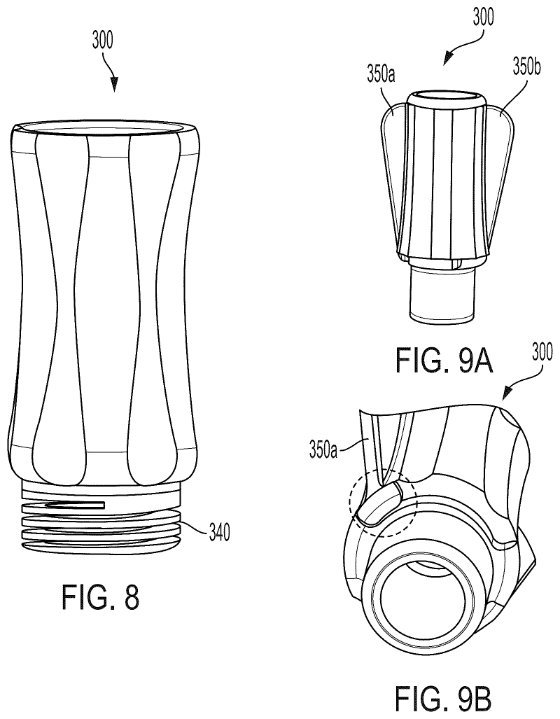

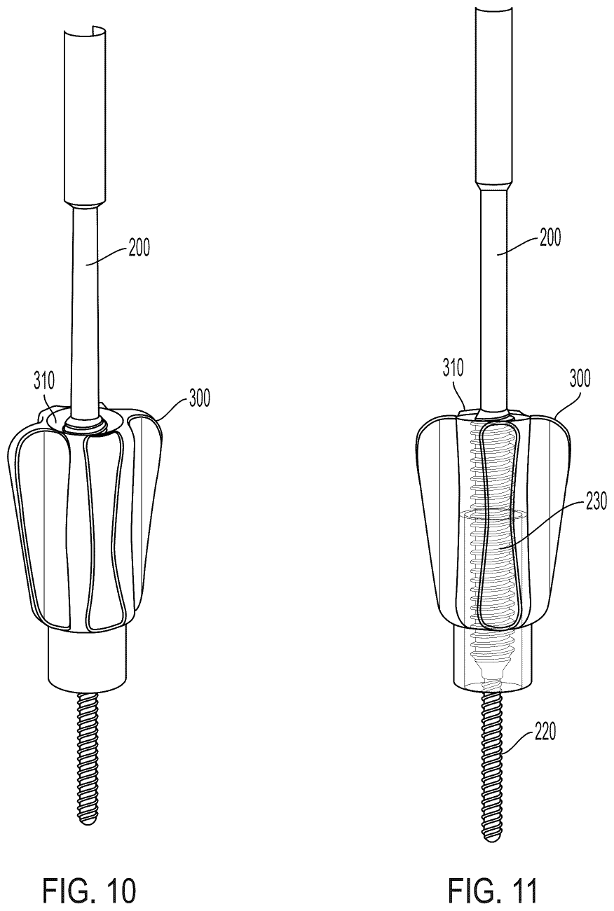

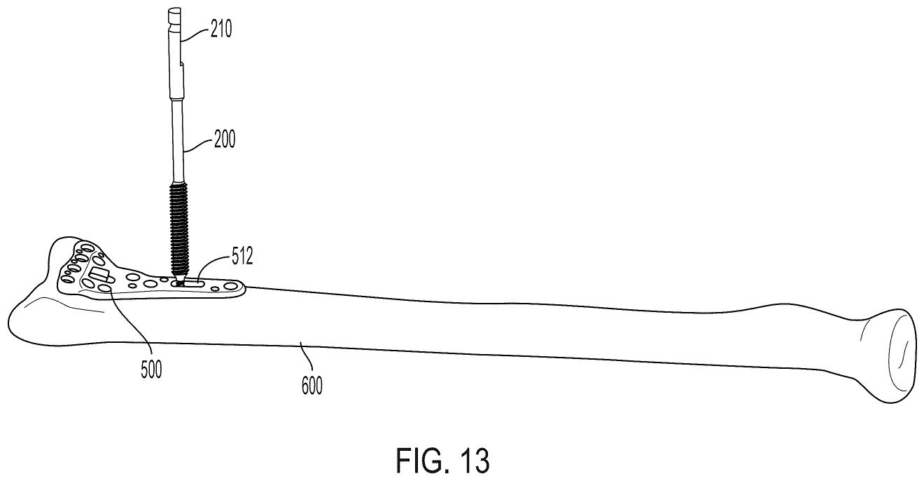

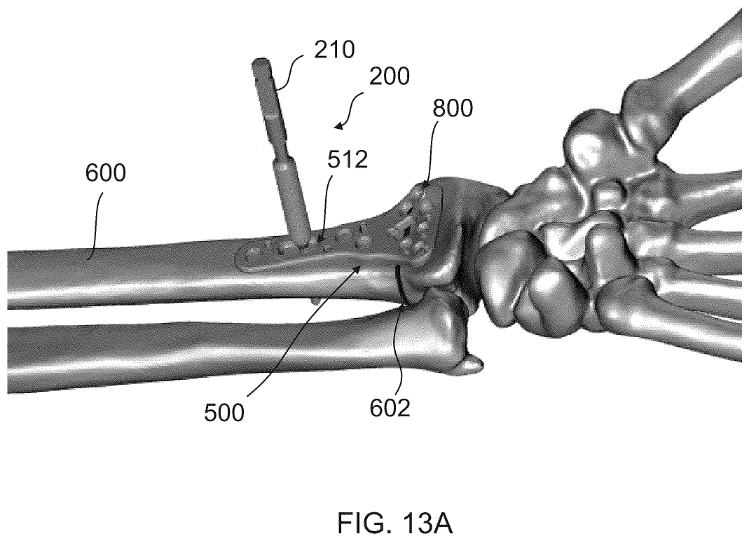

FIGS. 6 and 7 illustrate a body 300 according to an example of the present disclosure. The body 300 may include a bore 310 configured to circumscribe the rod, e.g., the rod 200 depicted in FIG. 5 (see, e.g., FIGS. 10 and 11). In some embodiments, the body 300 has a generally cylindrical shape, but it can be appreciated that any suitable shape may be used. In a particularly preferred embodiment, the length of the body 300 is longer than the width of the body 300. The body 300 disclosed herein, e.g., by having an elongate shape, may help a user provide sufficient torque to the body to provisionally fix a bone plate in place as will be described in more detail herein. However, in some embodiments, the length and the width of the body 300 are substantially the same or the length of the body 300 is shorter than the width of the body 300.

As shown in FIG. 6, in some examples, the body 300 includes one or more features (i.e. 320a, 320b, 320c) on an external surface of the body 300. The features 320a, 320b, 320c may provide texture to the body 300 so a user has better grip when translating the body 300 about the rod 200. In some embodiments, the features 320a, 320b, 320c may include one or more ridges, wings, indentations, or angled edges. While the depicted embodiment includes the one or more features 320a, 320b, 320c extending along a portion of the body 300, in some embodiments, the one or more features 320a, 320b, 320c extends along an entire length of the body 300. Referring to FIG. 7, one or more of the features 320c may protrude from the body 300 (e.g., from an inner wall of the body 300) a greater distance than the remainder of the one or more features 320a, 320b. This may give the user better leverage and allow the user to apply a greater torque to the body 300.

In some embodiments, a portion of the body 300, such as the proximal end portion 330, includes an external diameter that is smaller than the remainder of the body 300. For example, the body 300 may have a smaller profile on the proximal end portion 330 configured to engage with the bone plate. This may make it easier to translate the body 300 through tissue around the bone and bone plate without requiring a larger incision. In a particularly preferred embodiment, the proximal end portion 330 of the body 300 has a diameter substantially the same as a width of the bone plate to provide as much contact with the plate as possible while maintaining a smaller width. The larger diameter of the remainder of the body 300 may make it easier for a user to engage the body 300. However, in some embodiments, the diameter of the body 300 may be uniform along the length or the proximal end portion 330 may have a diameter greater than the diameter of the remainder of the body 300.

A shown in FIG. 7, in some examples, the bore 310 of the body 300 may include one or more diameters. For example, a portion 310a of the bore 310 may include a diameter D1 configured to interface with the rod 200. In some examples, the portion 310a of the bore 310 configured to interface with the rod 200 comprises a thread configured to interface with a thread of the rod 200. In some examples, a portion of the bore 310b may include a diameter D2 configured to receive a portion of the plier like device 400, such as an end portion of the first or second member 410, 420 (e.g., end portion 414 as shown in FIG. 2). The portion 310a may have a diameter (D1) smaller than the diameter of the portion 310b (D2). This allows the rod to pass through portion 310b and engage with portion 310a. In some embodiments, the diameter of portion 310a is substantially the same as the diameter of at least a portion of the rod 200, such as the second portion 230.

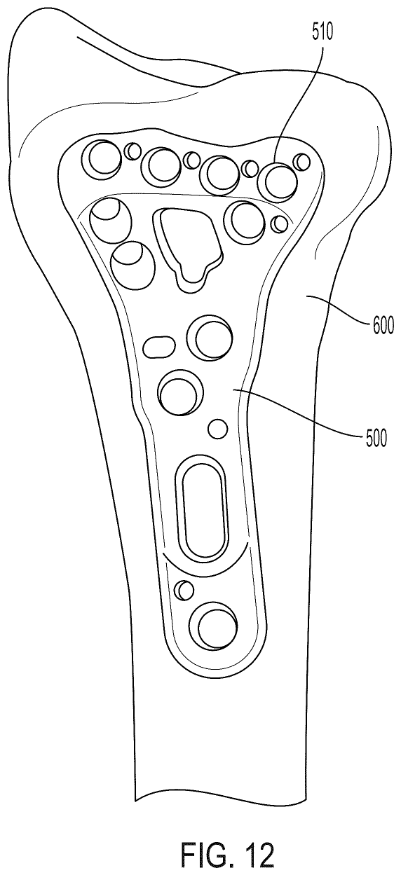

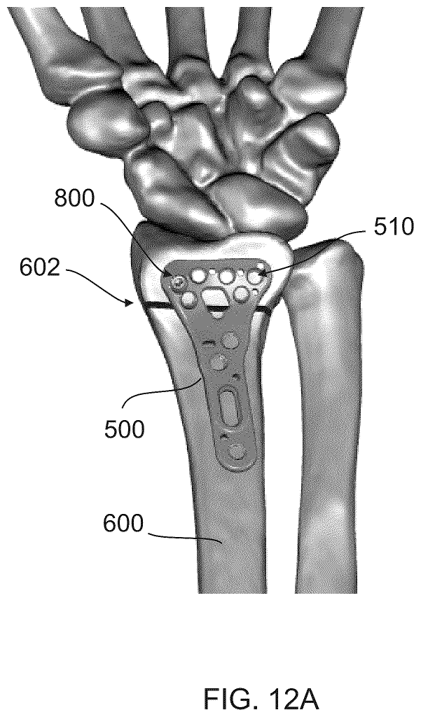

FIGS. 8, 9A, and 9B illustrate bodies 300 according to different or additional examples of the present disclosure. The examples of body 300 as shown in FIGS. 8, 9A, and 9B can incorporate any of the features of the body 300 as shown in FIGS. 6 and 7, except for the difference disclosed herein with reference to FIGS. 8, 9A, and 9B. The body 300 as shown in FIGS. 6 and 7 can incorporate any of the features of the examples of body 300 as shown in FIGS. 8, 9A, and 9B.

As depicted in FIG. 8, in some embodiments, an end (e.g., proximal end) of the body includes a mechanical spring 340. The mechanical spring 340 may exert a downward force on the bone plate and aid in compressing the bone plate against a surface of the bone. Additionally, the amount of force applied by the mechanical spring 340 may be adjusted by loosening and tightening the body 300 while contacting the surface of the bone plate.

As depicted in FIGS. 9A and 9B, in some embodiments, the body 300 may include adjustable wings 350a, 350b. The wings 350a, 350b may be able to rotate about the body 300 without movement or rotation of the body 300 itself. For example, as shown in FIG. 9B, the body 300 may include a track and the adjustable wings 350a, 350b may include a groove configured to translate along the track. Rotation of the wings 350a, 350b about the body 300 may only rotate the body 300 when the groove reaches the end of the track. This may allow a user to move the wings 350a, 350b out of the way, such as if the wings 350a, 350b are interfering with one or more holes of the bone plate.

FIGS. 10 and 11 illustrate the body 300 circumscribing the rod 200. The body 300 may be configured to translate up and down the rod 200. In some embodiments, the bore 310 of the body 300 may include one or more features configured to interface with one or more features of the rod 200 for translating the body 300 up and down the rod 200. In some embodiments, the one or more features of the body 300 may extend along the entire bore 310 or a portion of the bore 310 of the body 300. As depicted in the FIG. 11, in an example embodiment, the bore 310 of the body 300 may include a thread configured to interface with a thread of the rod 200. In this embodiment, rotation of the body 300 causes the body 300 to translate up and down the threads of the rod 200.

In some embodiments, the body 300 may be configured to only engage a portion of the rod 200, such as the second portion 230 of the rod 200. As discussed previously, in some embodiments, at least a portion of the bore 310 has substantially the same diameter as a portion of the rod 200. In some embodiments, the bore 310 of the body 300 is substantially the same or larger than the diameter of the rod 200. This may allow the rod 200 to translate through the bore 310 (e.g., the entire bore) of the body 300.

### Methods of Use

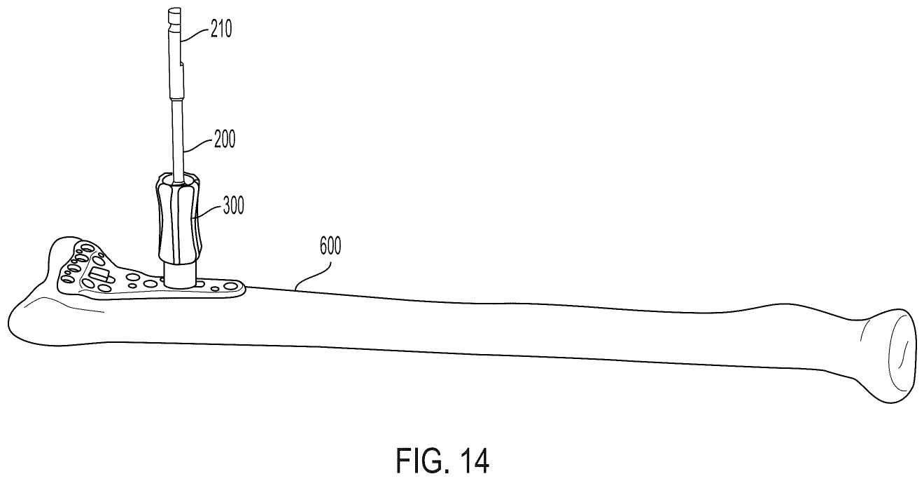

FIGS. 12, 12A, 13, 13A, 14, 14A, 15, and 15A illustrate certain steps of a method of aligning a bone fracture according to an example of the present disclosure. While FIGS. 12, 12A, 13, 13A, 14, 14A, 15, and 15A illustrate a radius bone, it can be appreciated that the system disclosed herein (e.g., system 100) may be used on any suitable bone fracture and/or on any bones and/or joints.

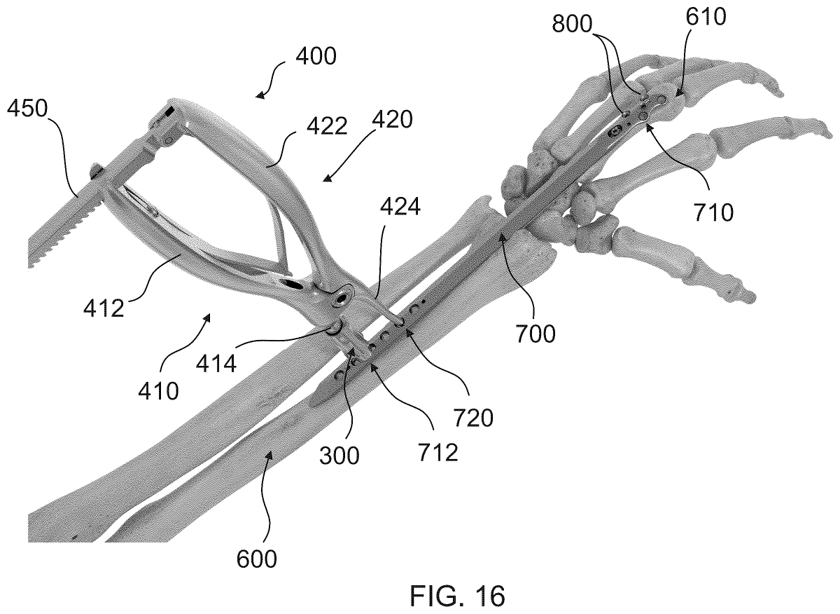

FIGS. 16 through 19 illustrate certain steps of additional methods of using the system disclosed herein. While FIGS. 16 through 19 illustrate a forearm including at least a radius bone, an ulna bone, a wrist joint, and metacarpals, it can be appreciated that the system disclosed herein may be used on any suitable bone fracture and/or on any bones and/or joints.

Any features of the configurations and/or methods of use of the rod, the plier like device, the body, the bone plate, and/or fasteners illustrated in FIGS. 12-19 can be incorporated into other configurations disclosed herein.

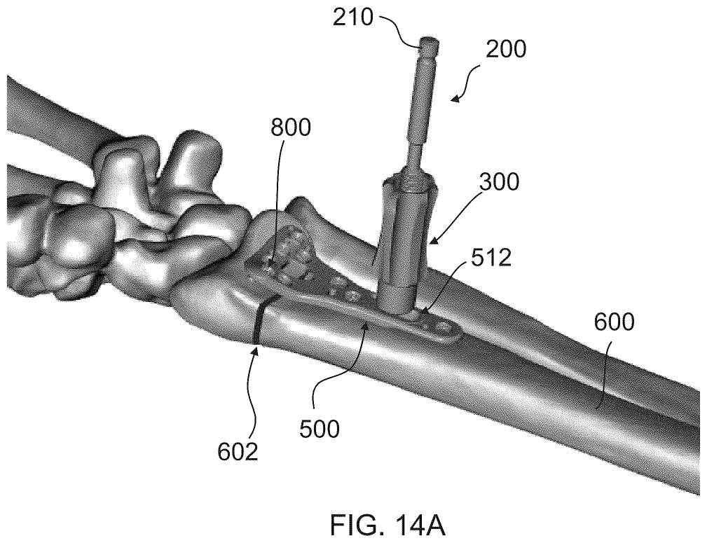

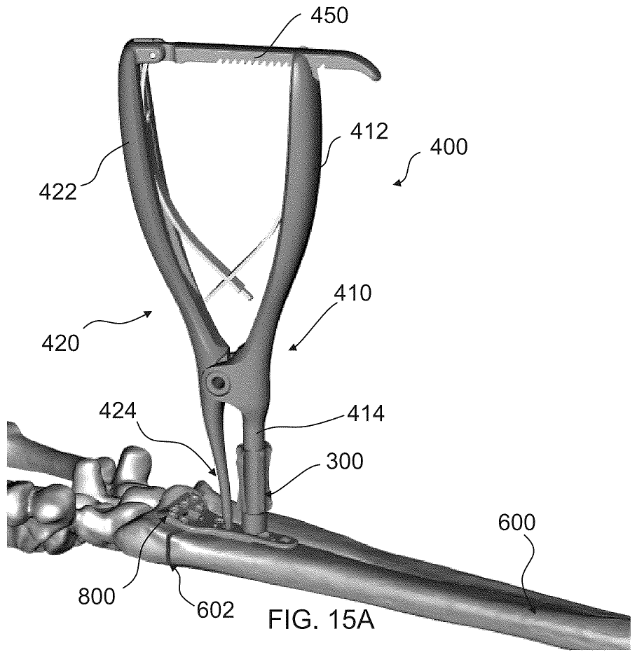

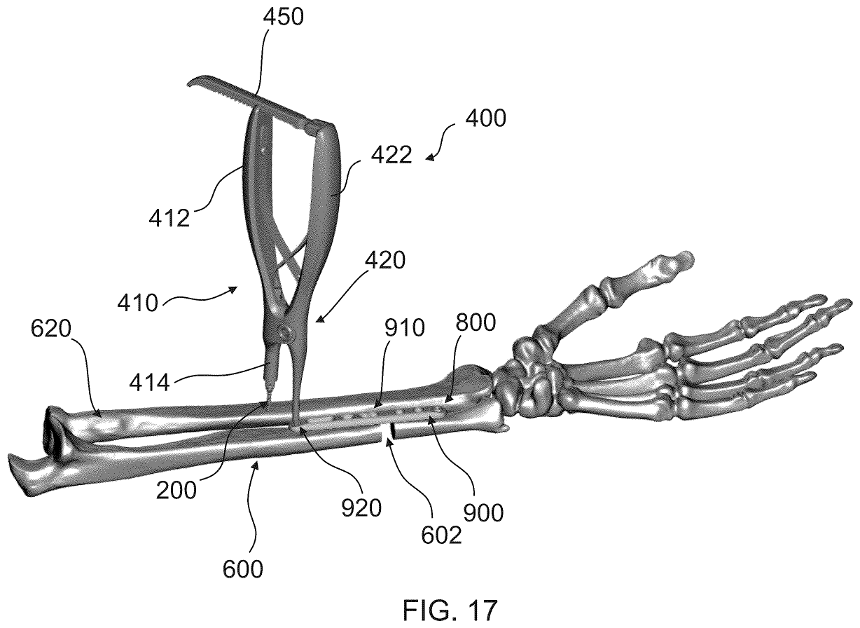

One or more steps as illustrated in FIGS. 12, 12A, 13, 13A, 14, and 14A, may be adapted and combined with any of the steps shown in FIGS. 15, 15A-19. For example, one or more of the methods disclosed herein may include positioning a bone plate along a fracture site of a bone as shown in FIGS. 12 and 12A, or across multiple bones. FIG. 12A illustrates the same bone plate 500 being placed over a bone 600 as FIG. 12, except that FIG. 12A illustrates an exemplary location and type of a bone fracture 602 and an example use of a screw 800 to achieve initial fixation of the bone plate to the bone. FIGS. 13A, 14A, 15A similarly show the exemplary bone fracture location and type 602 and the screw 800 but otherwise illustrate the same step as FIGS. 13, 14, and 15 respectively. In FIGS. 12, 13, 14, and 15, the location of the fracture may be more proximal or more distal, and/or the fracture may have a different shape and/or be of a different type as shown in FIGS. 12A, 13A, 14A, 15A. In some embodiments, the method may include positioning a bone plate 500, 900 along a fracture site of a bone 600 as shown in FIGS. 12 and 17. As shown, the bone plate 500, 900 can extend over bone fragments on opposite sides of the fracture 602. In FIGS. 16, 18, and 19, the method may include positioning a bone plate 700 along multiple bones. The choice of plate placement may be due to, e.g., location of the fracture site. In some embodiments, when the facture site is further away from the ends of a bone, the plate may be placed across two bone fragments across the fracture site. In some embodiments, when the fracture site is closer to or at an end of a bone, the plate may be placed over multiple bones, such as in wrist spanning to fix distal radius fractures. The bone plate 700 illustrates an example wrist spanning plate. Although bone plates 500, 700, 900 are illustrated, any other suitable bone plate can be used with the system disclosed herein.

The bone plate 500, 700, 900 may include one or more apertures 510, 710, 910 for receiving fasteners configured to secure the bone plate 500, 700, 900 to a bone, such as the bone 600 and another bone, such as bone 610. In some implementations, bone 600 may be a radius or an ulna. In some implementations, bone 610 may be a metacarpal or another suitable bone. In some embodiments, at least some of the apertures 510, 710, 910 may be circular. In some embodiments, at least some of the apertures 510, 710, 910 may be oblong (also referred to herein as slot(s)). In some embodiments, at least some of the apertures 510, 710, 910 may be slanted at an angle relative to the plate. For example, FIGS. 12A, 13A, 14A, 15A illustrates a screw 800 inserted into a slanted aperture 510 and into the bone 600. In some embodiments, the method may further include inserting a fastener (e.g., one or more screws 800 such as shown in FIGS. 13A and 16-19) in one of the apertures 510, 710, 910 of the bone plate 500, 700, 900 before alignment. This may create an initial point of fixation of the bone plate 500, 700, 900 to the bone, such as bone 600 or bone 610.

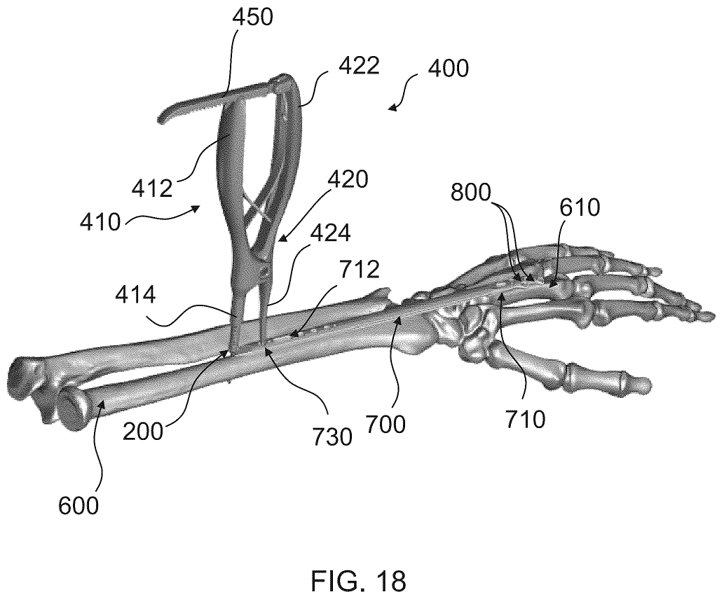

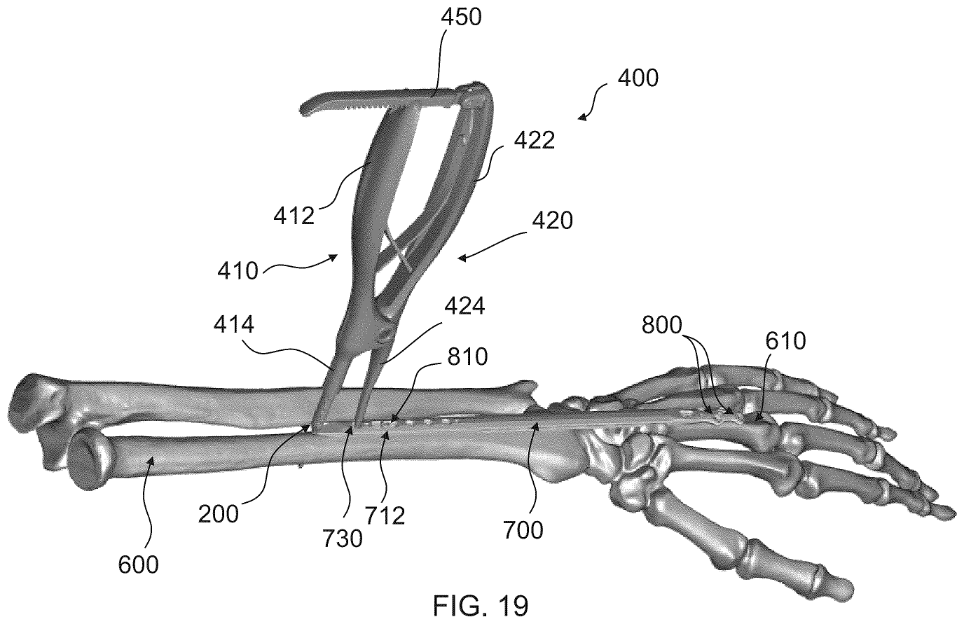

The method may include partially inserting the rod 200 into a portion of the bone 600 or another bone. Referring to FIGS. 13 and 13A, the method may include (partially) inserting the rod 200 into a portion of the bone 600. In some embodiments, the rod 200 is inserted into an additional aperture of the bone plate 500, 700, 900 such as a slot 512 (see FIGS. 13), 712 (see FIG. 16). In some embodiments, the rod 200 is inserted into one fragment of the bone fracture, that is, not through an aperture of the bone plate 500, 700, 900, while the fastener (e.g., one or more screws) is inserted into the bone plate 500, 700, 900 on another fragment of the bone fracture (such as shown in FIG. 17) or on the other bone 610 different from the bone 600 in which the rod 200 is inserted (such as shown in FIGS. 16, 18, 19). While FIGS. 13, 13A, and FIG. 16 show the rod 200 being inserted into slot 512, 712 of the bone plate 500, 700, in some embodiments, the rod 200 is inserted into a portion of the bone coupled to the bone plate 500, 700, 900 at a location proximal or distal to the bone plate 500, 700, 900 or at another location (e.g., lateral, medial, etc.). FIGS. 18 and 19 illustrate the rob 200 being inserted directly into the bone 600 proximal to the plate 700. The difference between FIGS. 18 and 19 is that in FIG. 19, an additional fastener (e.g., screw 810) is placed in a slot 712 of the bone plate 700 to help translation of the bones along the longitudinal axis of the plate 700. In FIG. 18, the bones are less constrained along the longitudinal axis of the plate 700 due to the lack of the additional fastener 810 in the slot 712. As another example, FIG. 17 further illustrates the rod 200 being inserted into a bone 620 that is not coupled to the bone plate 900 or not extended over by the bone plate 900. In some embodiments, the bone 620 in which the rod 200 is inserted into is the ulna while the bone plate 900 is coupled to fractured segments of the radius 600 by the fastener 800.

In some embodiments, a pilot hole may be drilled into the bone 600 or another bone 620 prior to inserting the rod 200. In some embodiments, the rod 200 may be self-drilling and may be drilled directly into the bone 600 or another bone 620. For example, a driver may interface with one end (e.g., distal end) 210 of the rod 200 for insertion into the bone 600. In some embodiments, the driver is a powered driver or a manual driver. A portion of the rod 200, such as the first portion 220, may be inserted into the bone 600 or another bone 620 so that the remainder of the rod 200 is extending out of the bone 600 or another bone 620. For example, less than half of, a third of, or a fourth of the rod 200 may be inserted into the bone 600 or another bone 620, while the remainder of the rod 200 extends from a surface of the bone 600 or another bone 620. In some embodiments, the first portion 220 (e.g., more than 75%, more than 80%, more than 90%, or an entirety of the first portion 220) of the rod 200 is inserted into the bone 600 or another bone 620 while the second portion 230 extends from the surface of the bone 600 or another bone 620. In some embodiments, a distal tip of the rod 200 may extend from the opposite surface of the bone 600 or another bone 620.

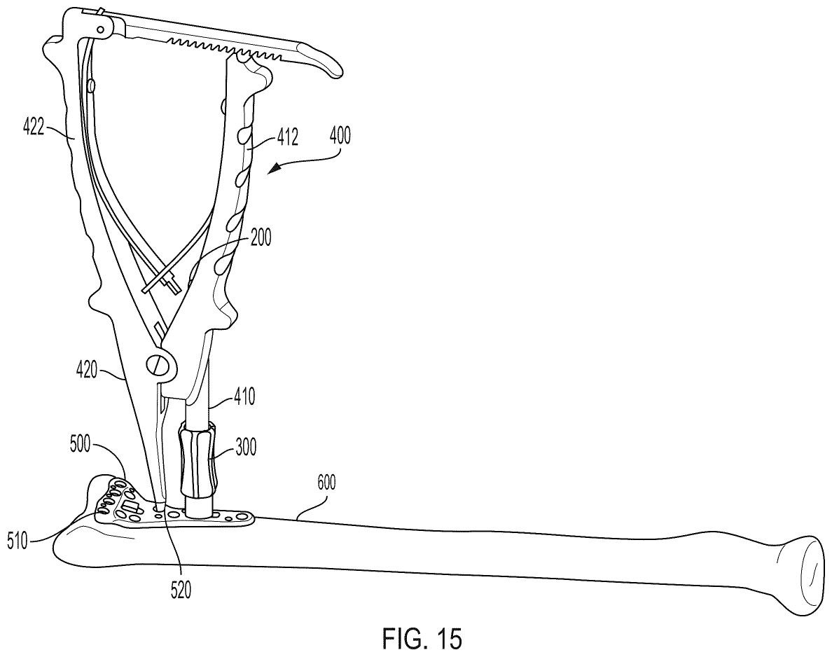

In some embodiments, such as when the rod 200 is inserted into a slot of the bone plate disclosed herein, the method may optionally include advancing the body 300 over the rod 200 so that the body 300 circumscribes a portion of the rod 200 as shown in FIG. 14, FIG. 14A, FIG. 15, FIG. 15A, and FIG. 16. The body 300 may be advanced over the rod 200 before or after the rod 200 is inserted into the bone 600. For example, if the body 300 is advanced over the rod 200 after the rod 200 is inserted into the bone 600, the body 300 may be placed over an end (e.g., distal end) portion of the rod 200, such as the driver interface 210. If the body 300 is advanced over the rod 200 before the rod 200 is drilled into the bone 600, the body 300 may be placed over either end of the rod 200, such as the first portion 220 or the driver interface 210. In some embodiments, the body 300 may engage a portion of the rod 200, such as the second portion 230 of the rod 200. As described previously or elsewhere in the present disclosure, in some embodiments, the rod 200 includes a thread configured to engage with a thread of the body 300. In some embodiments, other methods of holding the bone plate and the aligned bone fracture, such as k-wire, locking screw, clamp, etc., may be used, as will be described in more detail elsewhere in the present disclosure.

Referring to FIGS. 15, 15A-19, after the rod 200 has been inserted into the bone 600 or another bone 620 and/or optionally the body 300 is advanced over the rod 200, the method may include interfacing the plier like device 400 with the rod 200 and bone plate 500, 700, 900. For example, in some embodiments, the method includes placing the first member 410 of the plier like device 400 over rod 200. The channel 430 of the first member 410 may be placed over the second portion 230 of the rod 200 extending from the bone 600. In some embodiments, such as shown in FIGS. 15 and 16, the proximal end of the channel 430 may interface with a portion of the body 300. In some embodiments, the body 300 may include a recess configured to receive an end (e.g., proximal end) of the first member 410.

The method may further include positioning the second member 420 into a feature 520, 720, 920 of the bone plate 500, 700, 900. In some embodiments, the feature of the bone plate may include a bump, a blind hole, an aperture (e.g., a slot, a screw hole, a k-wire hole, a window), or any other suitable feature. In a particularly preferred embodiment, the feature 520, 720, 920 may include an aperture 510, 710, 910 of the bone plate 500, 700, 900. A user may engage an end (e.g., proximal end) portion of the second member 420 with an internal surface of the aperture before providing compression or distraction. The one or more cutouts or one or more ridges of the second member 420 (as disclosed herein elsewhere) may provide grip between the bone plate 500, 700, 900 and the second member 420. In some embodiments, the aperture of the bone plate 500, 700, 900 may include a thread. A user may engage an end (e.g., proximal end) portion of the second member 420 with the thread of the aperture.

In some embodiments, the second member 420 is positioned in a feature 520, 720, 920 of the bone plate 500, 700, 900 on a different side of the fracture from the rod 200, such as on a same side of the fracture as the fixation device (e.g., fastener as disclosed herein elsewhere). In some embodiments, the second member 420 is positioned in a feature 520, 720, 920 of the bone plate 500, 700, 900 on the same side of the fracture as the rod 200.

Once the plier like device 400 is positioned, the method may include moving the first end portions 412, 422 of the first and second members 410, 420 toward each other for distraction or compression. For example, movement of the first end portions 412, 422 may cause the bone fragments to align under the bone plate 500, 700, 900. In some embodiments, such as shown in FIGS. 15 and 16, during distraction or compression, the rod 200 may move the bone fragment and the rod 200 may translate through a slot in the bone plate 500, 700.

In some embodiments, distraction or compression is determined based on the direction the second member 420 travels relative to the bone fracture. For example, in some embodiments, the system 100 provides distraction when the second member 420 moves toward the bone fracture upon moving the first and second member toward each other. In some embodiments, the system 100 provides compression when the second member 420 moves away from the bone fracture upon moving the first and the second member toward each other.

Both distraction and compression may be required to align the same bone fracture in some embodiments. In some embodiments, the method may include providing one of distraction or compression to the bone fracture. The method may further include rotating the plier like device 400 about the rod 200 to move the second member 420 into a position so that the second member 420 now travels in an opposite direction relative to the bone fracture.

For example, in the depicted embodiments such as in FIGS. 15, 15A, 16, 18, and 19, the second member 420 is distal to the first member 410 and the rod 200, and the second member 420 is on a different side or same side of the bone fracture than the first member 410 and the rod 200. In these depicted embodiments such as in FIGS. 15, 15A, 16, 18, and 19, moving the handle portions 412, 422 of the first and the second members 410, 420 together can move the second member 420 towards the bone fracture. This can result in distraction of the fracture (i.e. moving the bone fragments away from each other). If a user then needed to compress the bone fracture, the user may remove the second member 420 from the feature 520, 720 of the bone plate 500, 700 and rotate the second member 420 (such as approximately 180°) about the rod 200. In some embodiments, the user may position the second member 420 in a feature of the bone plate proximal to the rod 200 and the first member 410. In other words, the user may position the second member 420 so that movement of the first and second members 410,420 together moves the second member 420 away from the bone fracture. This results in compression of the bone fracture (i.e. moving the bone fragments toward each other).

In some embodiments, moving the handle portions 412, 422 of the first and second members 410, 420 toward each other causes the ridge 452 of the member to translate along the ratcheting element 450 to hold the first member 410 and the second member 420 in position relative to each other. After a desired compression or distraction is achieved, the ratcheting element 450 may act to hold the first member 410 and the second member 420 in place so that the desired bone alignment is maintained. In some embodiments, the amount of distraction or compression may be determined from the one or more markings 454 on the ratcheting element 450 of the device 400, such as depicted in FIG. 3 when the first member 410 and the second member 420 are held in place.

The system 100 may also rotate the fragments of the bone fracture to obtain proper alignment. For example, while the first member 410 is disposed on the rod 200 and the second member 420 is positioned in a feature of the bone plate 500, 700, 900, a user may rotate the second member about the rod 200 or rotate the first member 410 about the second member 420. In some embodiments, rotation of the fragments occurs when the first member 410 and the second member 420 are fixed by the ratcheting element 450.

After a desired compression, distraction, or rotation is achieved, such as shown in FIGS. 15, 15A, and 16, the method may include translating the body 300 down the rod 200 (towards the bone 600) to compress the bone plate 500 against the bone 600. For example, a user may translate the body 300 down the rod 200 until an end of the body 300 contacts a top surface of the bone plate 500, 700, 900. In some embodiments, the aperture of the bone plate 500, 700, 900 with the rod 200 includes a counterbore configured to receive an end (e.g., proximal end) of the body 300. The user may then continue to translate the body 300 until the body 300 provides enough friction between the bone plate 500, 700, 900 and the bone 600. This may act to hold the bone plate 500, 700, 900 in the correct position with the proper bone alignment prior to full fixation of the bone plate 500, 700, 900 to the bone 600. In some embodiments, translating the body 300 down the rod 200 includes rotating the body 300 around the rod 200 so that the internal threads of the body 300 translate down the thread of the rod 200. However, it can be appreciated based on the disclosure herein that other mechanisms may be used to compress the body 300 against the bone plate 500.

In some embodiments, the method may include inserting a k-wire into the bone 600 through an aperture of the bone plate 500, 700, 900 to provide provisional fixation of the bone plate 500, 700, 900 prior to removal of the plier like device 400. Additionally or alternatively, the method may include inserting fixation devices (e.g., fasteners), such as one or more screws, into the apertures 510, 710, 910 of the bone plate 500, 700, 900 to fully secure the bone plate 500, 700, 900 to the bone 600. The fixation devices may be inserted prior to or after removal of the plier like device 400. In some embodiments, a clamp can be applied to hold the plate and bone disclosed herein in position before removal of the plier like device 400.

In some embodiments, the method includes removing the rod 200 from the bone 600 after the bone plate 500, 700, 900 is secured to the bone 600. In a preferred embodiment, the rod 200 may be replaced with a fixation device (e.g., any suitable fixation devices as disclosed herein elsewhere) after the rod 200 is removed from the bone 600. In some embodiments, the portion of the rod 200 inserted into the bone 600 has a diameter substantially the same or smaller than a diameter of the fixation device used to replace the rod 200. The temporary rod 200 allows a point of fixation in the bone 600 to provide distraction or compression without potentially damaging or stripping a screw more permanently placed in the bone plate 500, 700, 900.

### EMBODIMENTS

Various aspects of the subject matter described herein are set out in the following numbered embodiments:

Embodiment 1. A system for at least one of distraction, compression, or rotation of a bone fracture, the system comprising: a rod configured to be partially inserted into a bone; a body defining a bore configured to circumscribe a portion of the rod not inserted into the bone; and a first member and a second member joined at a fulcrum, wherein the first member defines a channel for receiving another portion of the rod.

Embodiment 2. The system of embodiment 1, wherein the rod comprises a first portion with a first diameter and a second portion with a second diameter, and wherein the first diameter is greater than the second diameter.

Embodiment 3. The system of embodiment 2, wherein at least one of the first and second portions of the rod comprise a thread.

Embodiment 4. The system of any one of embodiments 1-3, wherein the bore of the body comprises a thread, and wherein the thread of the bore is configured to engage with a thread of the rod such that rotation of the body causes the body to translate up and down or axially along the rod.

Embodiment 5. The system of any one of embodiments 1-4, wherein the first member and the second member each comprise a first end portion on one side of the fulcrum and a second end portion on another side of the fulcrum, wherein the first end portion comprises a handle, and wherein movement of the first end portions together cause the second end portions to move away from each other.

Embodiment 6. The system of embodiment 5, wherein the first end portions are in a plane at an angle relative to a plane of the second end portions.

Embodiment 7. The system of embodiment 6, wherein the plane of the first end portions is substantially perpendicular to a plane of the second end portions.

Embodiment 8. The system of embodiment 5, wherein the first end portions are in a same plane as the second end portions.

Embodiment 9. The system of any one of embodiments 1-8, wherein the rod comprises a first end portion and a second end portion, wherein the first end portion is configured to be inserted into the bone and wherein the second end portion comprises a driver interface.

Embodiment 10. The system of any one of embodiments 1-9 further comprising a biasing element disposed between the first member and the second member.

Embodiment 11. The system of embodiment 10, wherein the biasing element comprises a first biasing element and a second biasing element, wherein the first biasing element is coupled to the first member and the second biasing element is coupled to the second member, and wherein the first biasing element and the second biasing element meet between the first member and the second member.

Embodiment 12. The system of any one of embodiments 1-11, wherein one of the first member or the second member comprises a ratcheting element and wherein another member of the first member and the second member comprises a ridge, and wherein movement of the one member towards the other member causes the ridge to translate along the ratcheting element to hold the first member and the second member in position relative to each other.

Embodiment 13. The system of embodiment 12, wherein the ratcheting element comprises one or more markings indicative of an amount of distraction or compression.

Embodiment 14. The system of any one of embodiments 1-13, wherein a diameter of the rod at a first end is greater than a diameter of the rod at a second end.

Embodiment 15. The system of any one of embodiments 1-14, wherein a portion of the body comprises one or more features on an external surface that provide texture to the body.

Embodiment 16. The system of any one of embodiments 1-15, wherein an end portion of the second member comprises a taper.

Embodiment 17. The system of embodiment 16, wherein the end portion of the second member further comprises one or more ridges.

Embodiment 18. The system of any one of embodiments 1-17, wherein a length of the second member is greater than a length of the first member.

Embodiment 19. The system of any one of embodiments 1-18, wherein a width of the body is smaller than a length of the body.

Embodiment 20. The system of any one of embodiments 1-19, further comprising a bone plate and at least one fastener.

Embodiment 21. A method of at least one of compressing, distracting, or rotating a bone fracture using a system, the system comprising: a rod configured to be at least partially inserted into a bone; a body defining a bore configured to circumscribe a portion of the rod not inserted into the bone; a first member and a second member joined at a fulcrum, wherein the first member defines a channel for receiving at least a portion of the rod; and a bone plate comprising one or more apertures. The method comprises: inserting the rod into the bone; placing the first member over the rod; positioning the second member into a feature of the bone plate; moving the first member and the second member toward each other to compress and distract the bone fracture.

Embodiment 22. A method of at least one of compressing, distracting, or rotating a bone fracture using a system, the system comprising: a rod configured to be partially inserted into any bone; a first member and a second member joined at a fulcrum, wherein the first member defines a channel for receiving at least a portion of the rod not inserted into any bone; and a bone plate comprising one or more apertures. The method comprises: inserting a fastener through one of the one or more apertures of the bone plate into a first bone or a second bone; inserting the rod into the first bone or a third bone; placing the first member over the rod; positioning the second member into a feature of the bone plate; moving the first member and the second member toward each other to compress and distract the bone fracture; and maintaining alignment after compressing or distracting the bone fracture while removing the rod from the first bone or the third bone.

Embodiment 23. The method of embodiment 22, wherein the feature of the bone plate is selected from the group consisting of a bump, a blind hole, and one aperture of the one or more apertures of the bone plate.

Embodiment 24. The method of any one of embodiments 22 or 23, further comprising drilling a hole into the first or third bone before inserting the rod into the first or third bone and inserting the rod into the hole of the first or third bone.

Embodiment 25. The method of any one of embodiments 22 or 23, wherein the rod is self-drilling and wherein inserting the rod into the first or third bone comprises drilling the rod into the first or third bone using a power tool.

Embodiment 26. The method of any one of embodiments 22-25, wherein the rod is inserted into the first bone or the third bone via an additional aperture of the bone plate.

Embodiment 27. The method of embodiment 26, wherein the additional aperture comprises a slot.

Embodiment 28. The method of any one of embodiments 23-27, wherein the rod is inserted into the first bone away from the bone plate.

Embodiment 29. The method of any one of embodiments 23-28, wherein the feature is proximal or distal to where the rod is inserted and wherein moving the first member and the second member together compresses or distracts the bone fracture.

Embodiment 30. The method of any one of embodiments 22-29, wherein inserting the fastener through one of the one or more apertures of the bone plate into a first bone or a second bone is performed before compression or distraction.

Embodiment 31. The method of embodiment 30, wherein the aperture that the screw is inserted into is on a first side of the bone fracture and wherein the rod is inserted on a second side of the bone fracture.

Embodiment 32. The method of any one of embodiments 22-31, further comprising removing the rod after compression or distraction.

Embodiment 33. The method of any one of embodiments 22-32, further comprising replacing the rod with a locking screw after removing the rod.

Embodiment 34. The method of embodiment 33, wherein a diameter of the locking screw is larger than or substantially the same as a diameter of the rod.

Embodiment 35. The method of any one of embodiments 22-34, wherein the feature of the bone plate comprises a first aperture of the one or more apertures, and wherein positioning the second member into the feature comprises engaging an end portion of the second member with a portion of the first aperture.

Embodiment 36. The method of embodiment 34, wherein the first aperture comprises a thread, and wherein positioning the second member into the feature comprises engaging the end portion of the second member with the thread of the first aperture.

Embodiment 37. The method of any one of embodiments 22-36, wherein one of the first member or the second member comprises a ratcheting element and wherein another member of the first member and the second member comprises a ridge, and wherein moving the one member towards the other member causes the ridge to translate along the ratcheting element to hold the first member and the second member in position relative to each other.

Embodiment 38. The method of embodiment 37, wherein the ratcheting element comprises one or more markings indicative of an amount of compression or distraction, and wherein the method comprises moving the first and the second members towards each other until the ridge aligns with a marking of the one or more markings corresponding to a desired amount of distraction or compression.

Embodiment 39. The method of any one of embodiments 22-38, wherein maintaining alignment comprises advancing a body to press against the bone plate, the body defining a bore configured to circumscribe a portion of the rod not inserted into the bone and the body configured to translate axially along the rod.

Embodiment 40. The method of any one of embodiments 22-39, wherein maintaining alignment comprises: inserting a k-wire in a k-wire hole; inserting a screw into another one of the one or more apertures of the bone plate; or clamping the bone plate and the bone.

Embodiment 41. The method of any one of embodiments 22-40, wherein the first bone is radius, the second bone is a metacarpal, and the third bone is ulna.

Embodiment 42. The system or method of any one of preceding embodiments, wherein the bone plate is a plate for radius fracture

Embodiment 43. The system or method of any one of preceding embodiments, wherein the bone plate is a wrist spanning plate.

### Terminology

Although this disclosure has been described in the context of certain embodiments and examples, it will be understood by those skilled in the art that the disclosure extends beyond the specifically disclosed embodiments to other alternative embodiments and/or uses and obvious modifications and equivalents thereof. In addition, while several variations of the embodiments of the disclosure have been shown and described in detail, other modifications, which are within the scope of this disclosure, will be readily apparent to those of skill in the art. It is also contemplated that various combinations or sub-combinations of the specific features and aspects of the embodiments may be made and still fall within the scope of the disclosure. For example, features described above in connection with one embodiment can be used with a different embodiment described herein and the combination still fall within the scope of the disclosure. It should be understood that various features and aspects of the disclosed embodiments can be combined with, or substituted for, one another in order to form varying modes of the embodiments of the disclosure. Thus, it is intended that the scope of the disclosure herein should not be limited by the particular embodiments described above. Accordingly, unless otherwise stated, or unless clearly incompatible, each embodiment of this invention may comprise, additional to its essential features described herein, one or more features as described herein from each other embodiment of the invention disclosed herein.

Features, materials, characteristics, or groups described in conjunction with a particular aspect, embodiment, or example are to be understood to be applicable to any other aspect, embodiment or example described in this section or elsewhere in this specification unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The protection is not restricted to the details of any foregoing embodiments. The protection extends to any novel one, or any novel combination, of the features disclosed in this specification (including any accompanying claims, abstract and drawings), or to any novel one, or any novel combination, of the steps of any method or process so disclosed.

Furthermore, certain features that are described in this disclosure in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations, one or more features from a claimed combination can, in some cases, be excised from the combination, and the combination may be claimed as a subcombination or variation of a subcombination.

Moreover, while operations may be depicted in the drawings or described in the specification in a particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Further, the operations may be rearranged or reordered in other implementations. Those skilled in the art will appreciate that in some embodiments, the actual steps taken in the processes illustrated and/or disclosed may differ from those shown in the figures. Depending on the embodiment, certain of the steps described above may be removed, others may be added. Furthermore, the features and attributes of the specific embodiments disclosed above may be combined in different ways to form additional embodiments, all of which fall within the scope of the present disclosure. Also, the separation of various system components in the implementations described above should not be understood as requiring such separation in all implementations, and it should be understood that the described components and systems can generally be integrated together in a single product or packaged into multiple products.

For purposes of this disclosure, certain aspects, advantages, and novel features are described herein. Not necessarily all such advantages may be achieved in accordance with any particular embodiment. Thus, for example, those skilled in the art will recognize that the disclosure may be embodied or carried out in a manner that achieves one advantage or a group of advantages as taught herein without necessarily achieving other advantages as may be taught or suggested herein.

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without other input or prompting, whether these features, elements and/or steps are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or”means one, some, or all of the elements in the list.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount. As another example, in certain embodiments, the terms “generally parallel” and “substantially parallel” refer to a value, amount, or characteristic that departs from exactly parallel by less than or equal to 15 degrees, 10 degrees, 5 degrees, 3 degrees, 1 degree, 0.1 degree, or otherwise.

Any methods disclosed herein need not be performed in the order recited. The methods disclosed herein include certain actions taken by a practitioner; however, they can also include any third-party instruction of those actions, either expressly or by implication. For example, actions such as “inserting a screw” include “instructing insertion of a screw.”

While the above detailed description has shown, described, and pointed out novel features as applied to various embodiments, it can be understood that various omissions, substitutions, and changes in the form and details of the devices or algorithms illustrated can be made without departing from the spirit of the disclosure. As can be recognized, certain embodiments described herein can be embodied within a form that does not provide all of the features and benefits set forth herein, as some features can be used or practiced separately from others. The scope of certain embodiments disclosed herein is indicated by the appended claims rather than by the foregoing description. All changes which come within the meaning and range of equivalency of the claims are to be embraced within their scope.

What is claimed is:

1. A system for at least one of distraction, compression, or rotation of a bone fracture, the system comprising:
a rod configured to be partially inserted into a bone;
a body defining a bore configured to circumscribe a portion of the rod not inserted into the bone; and
a first member and a second member joined at a fulcrum, wherein the first member defines a channel for receiving another portion of the rod not inserted into the bone.

2. The system of claim 1, wherein the rod comprises a first portion with a first diameter and a second portion with a second diameter, and wherein the first diameter is greater than the second diameter.

3. The system of claim 2, wherein at least one of the first and second portions of the rod comprise a thread.

4. The system of claim 1, wherein the bore of the body comprises a thread, and wherein the thread of the bore is configured to engage with a thread of the rod such that rotation of the body causes the body to translate axially along the rod.

5. The system of claim 1, wherein the first member and the second member each comprise a first end portion on one side of the fulcrum and a second end portion on another side of the fulcrum, wherein the first end portion comprises a handle, and wherein movement of the first end portions together cause the second end portions to move away from each other.

6. The system of claim 5, wherein the first end portions are in a plane at an angle relative to a plane of the second end portions.

7. The system of claim 5, wherein the first end portions are in a same plane as the second end portions.

8. The system of claim 1, wherein the rod comprises a first end portion and a second end portion, wherein the first end portion is configured to be inserted into the bone and wherein the second end portion comprises a driver interface.

9. The system of claim 1, wherein one of the first member or the second member comprises a ratcheting element and wherein another member of the first member and the second member comprises a ridge, and wherein movement of the one member towards the other member causes the ridge to translate along the ratcheting element to hold the first member and the second member in position relative to each other.

10. The system of claim 9, wherein the ratcheting element comprises one or more markings indicative of an amount of distraction or compression.

11. The system of claim 1, wherein an end portion of the second member comprises a taper.

12. The system of claim 11, wherein the end portion of the second member further comprises one or more ridges.

13. The system of claim 1, wherein a length of the second member is greater than a length of the first member.

14. The system of claim 1, further comprising a bone plate and at least one fastener.

15. A method of at least one of compressing, distracting, or rotating a bone fracture using a system,
the system comprising:
a rod configured to be partially inserted into any bone;
a first member and a second member joined at a fulcrum, wherein the first member defines a channel for receiving at least a portion of the rod not inserted into any bone; and
a bone plate comprising one or more apertures, wherein the method comprises:
inserting a fastener through one of the one or more apertures of the bone plate into a first bone or a second bone;
inserting the rod into the first bone or a third bone;
placing the first member over the rod;
positioning the second member into a feature of the bone plate;
moving the first member and the second member toward each other to compress or distract the bone fracture; and
maintaining alignment after compressing or distracting the bone fracture while removing the rod from the first bone or the third bone.

16. The method of claim 15, wherein the feature of the bone plate is selected from the group consisting of a bump, a blind hole, and one aperture of the one or more apertures of the bone plate.

17. The method of claim 15, wherein the rod is inserted into the first bone or the third bone via an additional aperture of the bone plate.

18. The method of claim 17, wherein the additional aperture comprises a slot.

19. The method of claim 16, wherein the rod is inserted into the first bone away from the bone plate.

20. The method of claim 16, wherein the feature is proximal or distal to where the rod is inserted and wherein moving the first member and the second member together compresses or distracts the bone fracture.

21. The method of claim 15, wherein inserting the fastener through one of the one or more apertures of the bone plate into a first bone or a second bone is performed before compression or distraction.

22. The method of claim 21, wherein the aperture that the screw is inserted into is on a first side of the bone fracture and wherein the rod is inserted on a second side of the bone fracture.

23. The method of claim 15, wherein maintaining alignment comprises advancing a body to press against the bone plate, the body defining a bore configured to circumscribe a portion of the rod not inserted into the bone and the body configured to translate axially along the rod.
